(19) United States
(12) Patent Application Publication
(10) Pub. No.: US 2017/0281676 A1
(43) Pub. Date: Oct. 5, 2017

(54) HIV-CURE

(71) Applicant: Lupus Foundation of Gainesville, GAINESVILLE, FL (US)

(72) Inventor: Eftim Josif Adhami, Gainsville, FL (US)

(73) Assignee: Lupus Foundation of Gainesville, GAINESVILLE, FL (US)

(21) Appl. No.: 15/480,105

(22) Filed: Apr. 5, 2017

Related U.S. Application Data
(60) Provisional application No. 62/318,366, filed on Apr. 5, 2016.

(51) Int. Cl. A61K 33/24; A61K 31/047; A61K 31/415; A61K 31/336; A61K 33/00; A61M 16/12; A61K 9/00; A61M 5/172; A61M 16/10; A61M 16/04; A61M 16/08; A61K 31/045; A61K 31/5517

(52) U.S. Cl. CPC A61K 33/24; A61K 31/045; A61K 31/047; A61K 31/415; A61K 31/336; A61K 33/00; A61K 31/5517; A61K 9/0019; A61K 9/007; A61M 5/1723; A61M 16/104; A61M 16/04; A61M 16/085; A61M 16/0891; A61M 16/1005; A61M 16/122; A61M 2205/3303; A61M 2250/00; A61M 2230/432; A61M 2230/437; A61M 2205/502; A61M 2202/0208; A61M 2202/0283; A61M 2202/0241; A61M 2202/048

(57) ABSTRACT

The embodiments use an innovative approach with the goal of permanent eradication of the virus. Instead of using drugs that block different stages of the virus life cycle (which have failed to induce a permanent cure), or approaches attempting to track infected cells, the embodiments use small virucidal molecules that are known to destroy the virus in vitro and that can easily penetrate all human cells, including memory cells or other reservoir cells. The problem with the use of small molecules is their toxicity to humans or animals when administered in doses sufficient to achieve intracellular concentrations high enough to destroy the virus in all forms. The embodiments overcome these toxicities (especially the comatose state) by using a 24-hour treatment with general anesthesia, endotracheal intubation with hemodynamic support, and controlled monitored ventilation; also, a combination of these molecules are used which decreases toxicity but has additive virucidal effects.

### STATEMENT REGARDING FEDERALLY SPONSORED RESEARCH OR DEVELOPMENT

Not applicable

### FIELD OF THE EMBODIMENTS

The field of the embodiments is the treatment of Human Immunodeficiency Virus (HIV) infections.

### BACKGROUND OF THE EMBODIMENTS

HIV infection was one of the major public health challenges of the 20th century but it continued to be so in the beginning of our century. At the end of 2007 over 33 million people were living with HIV/AIDS. Over

1 million people were living with HIV/AIDS only within the United States by the end of 2003. Minorities have been overrepresented in HIV infection prevalence with African Americans accounting for 50% of all cases in 2005 (1). The combination antiretroviral therapy changed the life of millions of people by dramatically increasing life expectancy and improving quality of life, but it has not achieved a cure of the disease and brings to the table a number of adverse effects and drug interactions, as well as huge economic costs of a necessary lifetime therapy. The antiretroviral therapy usually reaches the goal of an undetectable viral load, but the virus replicates soon after interruption of this therapy, and it may even become more resistant (2). The source of this reactivation has been shown to be mostly the memory cells, especially resting CD4+ T cells, where the HIV provirus can persist for years. Also, low levels of replication continue even during the antiretroviral therapy (3). Thus, it is imperative, healthy and economic to find a way to eradicate the virus completely from the infected individuals.

Unfortunately, effective strategies to eliminate completely or separate the infected cells are not available (3). That is why this invention presented here is important: the inventor, Dr. Adhami, presents a new method that attempts to eradicate the virus by using small molecules that easily enter human cells of all kinds and have antiviral activity in vitro. They can kill the virus anywhere in the body, and they may be able to stop at least the low-grade replication of the virus that is not suppressed by the usual therapy. Trying to separate immunologically the infected cells from the non-infected cells may be more complex than using molecules that diffuse easily and have antiviral action. This idea is unique and has never been tested in animals or humans. On the other hand, these small molecules are usually toxic in concentrations that are necessary to be reached in order to have significant virucidal effects in cellular or extra-cellular locations. Dr. Adhami overcomes this problem by injecting the molecules after induction of general anesthesia in order to mitigate the toxic effects of these molecules, especially alcohols: e.g. induced coma can lead to aspiration of gastric contents and respiratory depression, but preventive sedation, intubation, mechanical ventilation and hemodynamic support, can control these adverse effects, just like we do when providing routine general anesthesia (4).

Differently from any other methods of treatment of HIV in the past, mostly medications, where the patient needs to take the medication daily for life, in this invention, the patient will usually need one-time treatment in an anesthesia setting, sufficient to clear the virus permanently from the body. This will be not only life-saving (especially for people with virus resistant to medications or with medication side effects) but also very cost-effective for society. It will also remove the social stigma of being HIV-positive from the individual. Even if repetitive treatments are required (no method is perfect), they are still done only a few times and there is no need for daily, expensive, lifetime medications.

Human Immunodeficiency Virus (HIV) infection was one of the most significant public health challenges encountered at the end of the 20th century and continues to be in this century the major lifelong viral illness known to humans. Despite the success of combined antiretroviral therapy in improving survival and quality of life of patients affected by HIV/AIDS, HIV remains a permanent infection and it is reactivated soon after antiretroviral therapy is discontinued, and sometimes it becomes resistant to this treatment. Thus, almost all the patients infected with the virus will need at some point in their life to take uninterrupted combined antiretroviral therapy. This brings to the table a number of potential side effects and huge economic costs to society. The virus is stored in many of the memory cells in latent state, but able to replicate again when these cells are activated. Also, even during antiretroviral therapy, there are low levels of viral replication that maintain another type of “reservoir”. Attempts to differentially destroy the infected reservoir cells or completely and permanently eradicate the virus from infected humans have all failed; one major issue is the difficulty to differentiate and isolate all the infected cells vs the non-infected cells.

In this invention, Dr. Adhami uses a special, new, completely radical and innovative approach with the goal of permanent eradication of the virus. Instead of using drugs that block different stages of the virus life cycle (which have failed to induce a permanent cure), or approaches attempting to track infected cells, Dr. Adhami is using small virucidal (or virus-killing) molecules that are known to destroy the virus in vitro and they can penetrate easily all human cells, including memory cells or any other reservoir cells.

This approach has never been tested in the past and can be used to eradicate other viruses that permanently invade human or animal organisms. The problem with the use of small molecules is their toxicity to humans (or animals) when administered in doses sufficient to achieve intracellular concentrations high enough to destroy the virus(es) in all forms. Dr. Adhami overcomes these toxicities (especially the comatose state) by using a single treatment with these molecules under general anesthesia and endotracheal intubation with hemodynamic support and controlled and monitored ventilation; also, he uses combinations of these molecules, a method that decreases toxicity but has additive virucidal effects.

### SUMMARY OF THE EMBODIMENTS

The embodiments of the HIV Cure are comprised of a special, new, completely radical and innovative approach with the goal of permanent eradication of the virus. Instead of using drugs that block different stages of the virus life cycle (which have failed to induce a permanent cure), or approaches attempting to track infected cells, Dr. Adhami is using small virucidal molecules that are known to destroy the virus in vitro and they can penetrate easily all human cells, including memory cells or any other reservoir cells. The problem with the use of small molecules is their toxicity to humans or animals when administered in doses sufficient to achieve intracellular concentrations high enough to destroy the virus in all forms. Dr. Adhami overcomes these toxicities (especially the comatose state) by using a 24-hour treatment with these molecules under general anesthesia and endotracheal intubation with hemodynamic support and controlled and monitored ventilation; also, he uses a combination of these molecules which decreases toxicity but has additive viricidal effects.

Thus, the general purpose of the invention is to provide a permanent destruction of HIV or other viruses with a single treatment procedure that lasts from several hours to 24 hours, after using IV infusions of small molecules with the ability to penetrate all body fluids and cells and destroy the virus(es) wherever they are.

The HIV infection was one of the most significant public health challenges encountered at the end of the 20th century and continues to be in this century the major lifelong viral illness known to humans. Despite the success of combined antiretroviral therapy in improving survival and quality of life of patients affected by HIV/AIDS, HIV remains a permanent infection and it is reactivated soon after antiretroviral therapy is discontinued, and sometimes it becomes resistant to this treatment. Thus, almost all the patients infected with the virus will need at some point in their life to take uninterrupted combined antiretroviral therapy.

This brings to the table a number of potential side effects and huge economic costs to society. The virus is stored in many of the memory cells in latent state, but able to replicate again when these cells are activated. Also, even during antiretroviral therapy, there are low levels of viral replication that maintain another type of “reservoir”. Attempts to differentially destroy the infected reservoir cells or completely and permanently eradicate the virus from infected humans have all failed; one major issue is the difficulty to differentiate and isolate all the infected cells vs the non-infected cells.

There has thus been outlined, rather broadly, the more important features of the embodiments of the HIV Cure in order that the detailed description thereof that follows may be better understood, and in order that the present contribution to the art may be better appreciated. There are, of course, additional features of the embodiments that will be described hereinafter and which will form the subject matter of the claims appended hereto.

In this respect, before explaining at least one embodiment of the embodiments in detail, it is to be understood that the embodiment is not limited in this application to the details of construction and to the arrangements of the components set forth in the following description or illustrated in the drawings. The embodiment or embodiments are capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be used as a basis for the designing of other structures, methods and systems for carrying out the several purposes of the embodiments. Additional benefits and advantages of the embodiments will become apparent in those skilled in the art to which the present embodiments relate from the subsequent description of the preferred embodiment and the appended claims, taken in conjunction with the accompanying drawings. It is important, therefore, that the claims be regarded as including such equivalent constructions insofar as they do not depart from the spirit and scope of the embodiments.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the embodiments of the application which is measured by the claims, nor is it intended to be limiting as to the scope of the embodiments in any way.

### BRIEF DESCRIPTION OF THE DRAWINGS

FIG. 1 is a schematic of an embodiment of the HIV-Cure showing the relationship of elements of the embodiments relative to the patient.

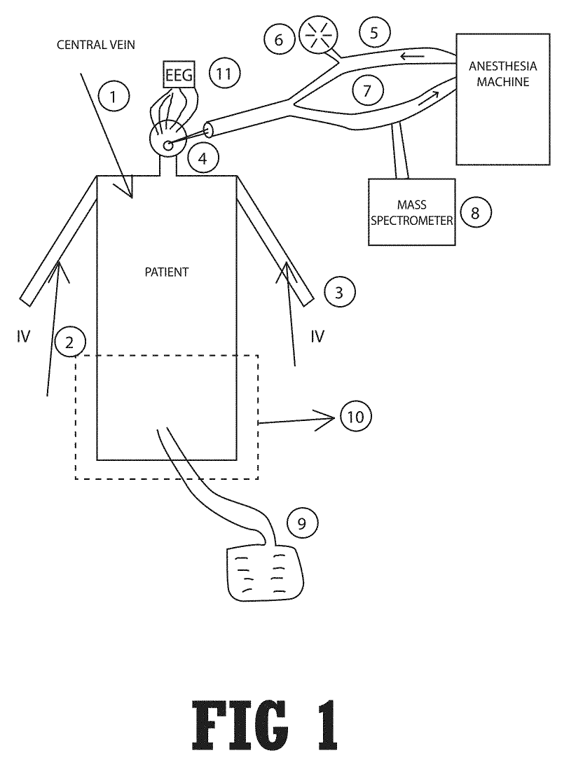

FIG. 2 is a schematic of an embodiment of the HIV-Cure showing the recordation of chemical levels.

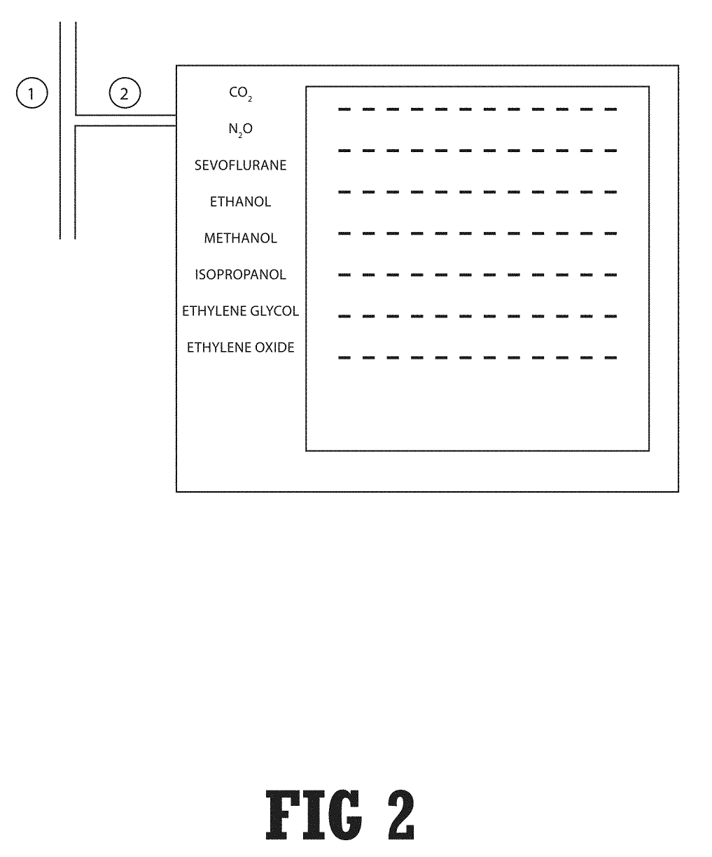

FIG. 3 is a schematic of an embodiment of the HIV-Cure showing the mechanism of controlling flow of gases.

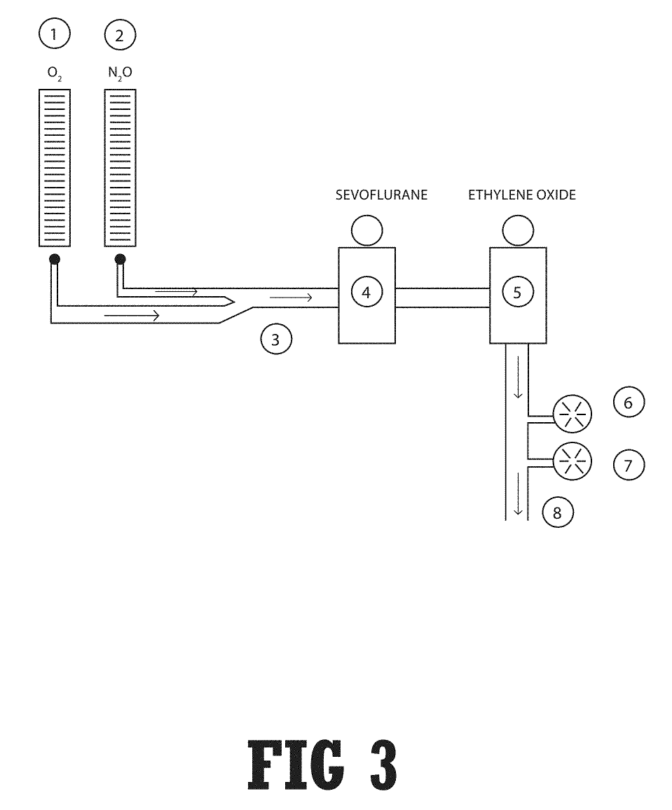

FIG. 4 is a schematic of an embodiment of the HIV-Cure showing the a decision tree analysis of the embodiment method.

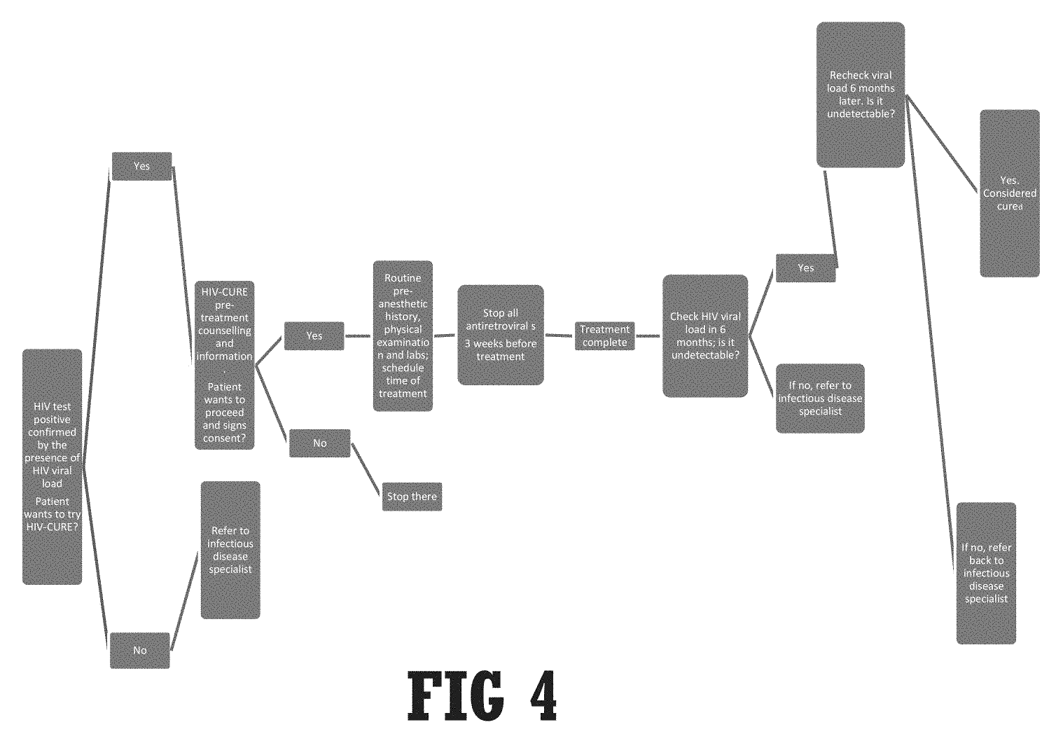

### DETAILED DESCRIPTION OF THE PREFERRED EMBODIMENTS

General Anesthesia

Before the treatment, each patient (human or animal, or their surrogate) who intends to undergo treatment for permanent eradication of HIV (or other viruses behaving similarly), agrees to the treatment with a standard anesthetic and treatment consent as it is customarily done in human or veterinary medicine clinics or hospitals (4). A standard pre-anesthetic screening and examination is done as it is usual for any surgical procedure. The patient is anesthetized and monitored for a period of maximum time of 24 hours. The 24-hour period is appropriate and it is calculated in order to give enough time for virus destruction, for the infusions to be administered according to the models below etc., but the actual time during the application may be slightly shorter or longer depending on the individual patient. The patient is placed on the anesthetic table, a mixture of nitrous oxide and oxygen in 2:1 ratio, together with 1-3% sevoflurane is administered. The concentrations of gases are administered through standard human or animal anesthesia machines and are adjusted according to standard anesthesia monitoring parameters (4).

Monitoring includes at a minimum: capnometry, oxygen saturation, blood pressure, heart rate, respiratory rate and clinical observation. After the induction of anesthesia, at least 2 peripheral IV lines are placed, paralysis is caused by using a standard muscle relaxer like vecuronium, rocuronium, pancuronium, etc, patient is intubated and placed on an automatic ventilator with a tidal volume of 10 ml/kg and respiratory rate adjusted according to the end-tidal carbon dioxide. The effects of muscle paralyzers are monitored with a neuro-stimulator. Then, the nitrous oxide is stopped and the sevoflurane concentration is increased as tolerated, in order to maintain deep anesthetic levels with sevoflurane and oxygen, if possible, without the need for nitrous oxide, achieving in this way high concentrations of oxygen in the breathing mixture; the purpose of high oxygen concentration is to mitigate some of the side effects of antiviral substances that are used in later stages, like alcohol-induced acidosis and ethylene oxide gas in the other model (see below). The treatment team tries to maintain slightly low concentrations of end-tidal carbon dioxide (30-35 mmHg), in order to induce mild alkalosis which is needed to avoid some of the side effects of alcohols (acidosis) (see below). The use of alkalosis induced by increasing minute ventilation, in order to counteract an acidotic state, is a common anesthetic practice (4). A central vein is placed for some of the alcohol infusions, if they are used later, either jugular or subclavian access. The central vein is necessary for ethanol infusions, because ethanol, one of the alcohols used, irritates peripheral veins (5). After the venous access is obtained, a continuous infusion of two solutions (50/50) of 5% dextrose and Ringer's lactate is started; the total infusion rate (both fluids added together) is set initially at 4 ml/kg/hour of anesthetic time and then adjusted according to urine output. Urine output is monitored with a small bladder catheter and is maintained at a minimum of 0.5 ml/kg/hour. The body temperature is monitored with a rectal probe and maintained normal with a warming thermostatic blanket. A solution of propofol can be used as an infusion if the monitoring data are stable. This either supplements the inhalation anesthesia or is used as a total intravenous anesthetic, depending on the monitoring anesthetic parameters or anesthesia expertise of the treatment team. The purpose of each anesthetic protocol is to maintain the patient with stable hemodynamic, ventilation, and metabolic parameters for up to 24 hours under general anesthesia and then the patient must be extubated and recover from anesthesia without complications. This is in accordance with usual anesthetic principles (4).

All patients treated with the current invention must have an HIV viral load measured in the blood a few days before the procedure to confirm HIV status (2), and they must stop the HIV medications 2-3 weeks before the treatment, in order to avoid any side effects from interaction of these medications with the small molecules/alcohols and anesthetics used during this treatment (2). A permanent undetectable HIV viral load is the goal of any method that tries to cure the HIV (3). Many chemical compounds that have small-size molecules, have been tested and can deactivate the HIV and other viruses in vitro and some are used as sterilants and disinfectants (6). The problem with these compounds is toxicity to human and animal cells when injected in concentrations able to kill the virus(es) (7, 8, 9). This invention addresses specifically the way how to mitigate different toxicities for each compound and how to use them for the full eradication of HIV or other viruses while, in the same time, controlling specific toxic effects of each chemical.

The main substances used in this invention are alcohols, especially ethanol, isopropyl alcohol (isopropanol), methanol, and ethylene glycol. These alcohols have been shown to inactivate the HIV (5, 10, 11, 12) and Feline Immunodeficiency Virus (FIV) (the cat counterpart of human HIV) viruses in vitro by denaturing their molecules. FIV has been accepted as a good model of human HIV and has very similar viral characteristics (13). But when administered in vivo, these alcohols have side effects specific to each one, especially when high concentrations (similar to external use) are reached in serum (7, 8, 9). These are all small molecules and are soluble in water and fat, and can penetrate in all human fluids and cells, they can penetrate lipid bilayers. They are distributed in all areas of watery body fluids, achieving concentrations similar to serum in these fluids (6).

They can also penetrate more in cells that replicate faster, like the cells infected with HIV (13, 14). As such, they can be effective in inactivating and destroying the FIV or HIV virus, or other viruses in at least some of the reservoirs. They can also penetrate into the central nervous system (14) and destroy the viral particles that cause neurological complications. The effects of alcohols in vivo on the body components or viruses themselves depend on the blood levels, just like any other substance, but also on the penetration of the substances from the blood to the interstitial fluids and then to cells and cytoplasm and nucleus.

Alcohols have been shown to penetrate all these barriers due to their dual lipid and water solubility. As such, this quality gives them high penetration capability and ability to kill the virus(es) in all tissues and fluids and cure the patient permanently, if sufficient concentrations are reached, and as long as the toxicities are controlled. Many studies have examined the effects of alcohol use (mainly ethanol) on the HIV virus and disease. Most of these data have shown negative health effects of chronic alcohol use in people with HIV/AIDS (1). On the other hand, in the current invention, the negative effects of ethanol when used chronically are actually beneficial for this acute model of HIV treatment. Some of these effects are listed below:

Ethanol and other alcohols have very high penetrating ability in all tissues (6). In chronic alcohol users this causes more negative health effects by affecting multiple organ systems. In the acute invention model, this is an advantage, because it creates high concentrations of alcohols in all the fluids and cells where HIV (or other viruses) are found and helps to inactivate them.

Ethanol was shown to increase HIV virus replication in peripheral blood lymphocytes (14) and in central nervous system (15, 16). These are deleterious effects in the chronic phase, because they increase viral burden and make the disease progress faster. But in the acute model of this invention, the goal is to expose all viral particles in the body to high alcohol concentrations and to destroy them. A major problem for current science that does not allow a complete cure of HIV, are the so-called “reservoirs” (3). These reservoirs have included CD4+ memory cells, lymphoid tissue of the gut, peripheral blood, bone marrow and the brain cells (17). Here the virus is latent and does not replicate and remains as a reservoir for future, so it escapes the antivirals; so, scientists have tried several methods to activate the latent virus (18), which have been until now unsuccessful in curing the disease. In the model of this invention, ethanol itself, not only increases the viral replication (14, 15, 16, 19), but also strongly potentiates apoptosis of reservoir cells (20), which exposes the latent viral particles to the extracellular fluid and makes them susceptible to be inactivated by ethanol and other virucidal molecules used in this invention. On the other hand, in the chronic HIV carrier and alcohol user, this effect makes the virus to spread to new cells and, as opposed to the acute model of the invention, it is deleterious and not beneficial.

In all stages of HIV infection, monocytes are infected with the virus, and they can cross the blood-tissue barrier and differentiate into macrophages (21). These macrophages seem to be very important latent reservoirs of HIV in patients on regular antiretroviral therapy, even more critical in preventing a complete cure as compared to CD4+ T cells, and this makes them the major reservoirs (21). Chen et al. (22) studied the effect of ethanol, and its metabolites: acetaldehyde and acetate, on monocyte function, by using a human macrophage hybridoma cell line and primary monocytes. The authors treated these cells with ethanol in 3 different concentrations in mM units. Converted by the inventor to clinical scale levels, these levels were equivalent to 115, 345, and 690 mg/dl. These are similar to the clinically achievable levels and to the levels used in this invention (around 400 mg/dl but can be higher since toxicity is monitored). They treated the cells with ethanol for 16 hours. This time period is also similar to the approximate treatment time of the invention (24 hours median but can be higher or lower as described above). This time is in hours and not in seconds or minutes as advocated by disinfectant use of alcohols. After treatment with ethanol, the cells were infected with HIV-1 virus. Ethanol in these concentrations did not affect the viability of the cells (so toxicity was low), but had immunosuppressive effects in all the immunogenic tests of these cells. This effect is desirable for the acute invention model to activate the latent virus. Similar effects were found for the metabolites of ethanol, acetaldehyde and acetate which were tested in similar molar concentrations like ethanol.

After inducing general anesthesia and reaching stable levels and inserting IV lines (as described above in the anesthetic model), the treatment team starts injecting first (in a central vein), an ethanol solution at a concentration of 10% (volume/volume). The loading dose is 10 ml/kg, which usually raises the serum concentration by about 100 mg/dL in humans. The dose is slowly injected over 60 minutes. A continuous baseline infusion of 1 mL/kg/hour is started in the same time, to maintain constant blood levels; but other boluses of 10 ml/kg are injected until the target level of 400 mg/dl is achieved. These infusion rates are the ones used in cases of ethanol IV injection for the treatment of poisonings by other alcohols and have been clinically tested as safe, if monitoring is appropriate (5, 6, 8). The target level of 400 mg/dl is the usual starting of a comatose state in a patient who is not a chronic drinker (23). Higher levels may be needed during the application of this invention and individualized to the patient, and the treatment sessions may be repeated a few months later if the viral load remains detectable. In this setting, where the potential side effects are controlled by general anesthesia, levels even higher, as illustrated in the case below (Adhami, E., unpublished). The purpose is to reach as high blood levels as possible, so that the levels in tissues and reservoir cells are high enough to destroy the virus, but without making the toxicity unmanageable. The dose of boluses and infusion rates are the same as those used clinically for other ethanol indications. Ethanol is already used as a treatment drug for several indications: local disinfection for bacteria and viruses, to kill tumors, for alcohol withdrawal, etc; these infusion rates are known to be used for treatment of toxicity of other alcohols, as an antidote (5). The use for the complete eradication of HIV and other viruses as described in this invention is completely a new indication for ethanol as a drug. The usual toxic effects of ethanol at these levels (400 mg/dl or above) in humans, are: 1. Lethargy and coma which can cause aspiration; people who are heavily drunk and fall in alcoholic coma can aspirate their gastric contents into the lungs and can be harmed by respiratory aspiration which can cause hypoxemia and death (5, 6, 9, 23, 24); the invention eliminates this negative effect by having the patient under general anesthesia and intubated to protect the airway before the alcohol infusion is started; this prevents also the effects of respiratory depression caused by ethanol seen in intoxicated individuals without an established airway; the ventilation in the patient here is already supported by endotracheal tube and ventilator (4). 2. If the ethanol-induced coma is very deep, it may have late deleterious effects on the brain that can be seen after awakening (Adhami, E., unpublished); ethanol can also cause some mild cerebral edema (24). The edema effect is mitigated by maintaining a mild alkalosis through hyperventilation during anesthesia, as described above (4). The invention mitigates the deep coma by injecting the antidote flumazenil, which counteracts the sedative effects of alcohol but not the antiviral effect. This antidote is used commonly to treat people with comas of different etiologies, but especially to counteract the effects of benzodiazepines. Ethanol and benzodiazepines share the brain receptors causing coma (6, 25). So, the use of flumazenil helps to make the ethanol-induced coma lighter, not as severe, but does not have any effect on the antiviral effect of alcohol. This is a new drug indication for flumazenil. Flumazenil is injected 5 times during the 24-hour period of anesthesia, each time with a dose of 0.01 mg/kg injected at a rate of 0.1 mg/min, similar to other clinical indications but adjusted to the time of this procedure (5, 6). 3. Hypoglycemia: this is a problem seen frequently in ethanol abusers or users, and during ethanol acute intoxication (24); the invention eliminates this by already having in place a continuous 5% dextrose infusion as described above 4. Hypovolemia: people using alcohol frequently get dehydrated and have hypovolemia due to diuretic effects of ethanol (25).

This is prevented by Ringer's lactate infusion as described above. A good hydration prevents hypotension and cardiovascular compromise during the treatment. 5. Hypothermia is commonly caused by alcohol intoxication (5, 24) and is eliminated in this model by using the automatic warming blanket as described above.

Ethanol serum levels are measured by gas chromatography (6) at least every 3 hours during the experiment and as needed, to monitor them and achieve the desired levels and then maintain constant. Alternative method is monitoring of end-expiratory levels of ethanol which correlate with the blood levels (see below and in diagrams). Towards the end of the 24-hour period, the infusions are stopped and anesthesia reversed in the usual way, the patient is sent to the recovery room to wake up and be monitored until fully awake and hemodynamically stable, as it is usually done for patients who complete different surgical procedures (4). The HIV viral load (or other viruses) is checked in 6 months and 1 year, to make sure that the virus never replicates again.

In an actual patient case managed by the inventor, a full cycle of the above model was seen to work successfully, even though it was tested unintentionally (Adhami E., unpublished). A 27-year-old male was brought to the emergency room by ambulance. The inventor was called to intubate for respiratory support. The patient was in coma with low respiratory rate and non-reactive. He was intubated with propofol and admitted to ICU. His labs showed ethanol blood level of 700 mg/dl. His history was unknown, but hospital records showed that he saw an internist as an outpatient 1 year earlier and was HIV positive. His regular labs at that time were all normal, including CD4+ cells in absolute numbers. The HIV viral load was 60,000 copies per ml at that time. He did not have any more records until this admission and he never took antiretroviral therapy. During the ICU stay, he was maintained in coma with propofol infusions and some opioid analgesics and muscle paralyzers, intubated and mechanically ventilated. Fluids given were mostly Ringer's lactate and dextrose 5% in water. Due to lack of history, the ICU staff assumed he was a chronic alcoholic. So, when patient's level of coma became lighter in the following days and patient developed mild tachycardia or mildly increased blood pressure, they thought it is a feature of alcohol withdrawal, which can occur and be dangerous during ICU stay for chronic alcoholics. So, they treated the patient with ethanol infusion, trying to decrease the level slowly over days. The next day, the blood level was 300 mg/dl. Patient recovered uneventfully after 10 days when he was extubated. At that time, his friends showed up and gave some history of the incident: the patient never drank alcohol, he was not a drinker, but he drank a lot that day while they were doing a drinking competition in a party. So, he did not have tolerance to alcohol.

After discharge, patient was lost to follow up. But he saw the same internist 2 years later and records of that encounter showed normal blood tests, including absolute CD4+ numbers. What was remarkable in this case, was that the HIV viral load this time was undetectable. Patient had never started any antiretroviral medication. This shows a case where the entire cycle of this invention worked. Without any treatment, it would be very unlikely that the viral load would drop from 60,000 to undetectable. Clinical data have shown that, if the viral load is over 50,000, the chances of full-blown AIDS without treatment in 5 years are 67% (2). This occurs only with steady increases in viral load over time, and not decreases.

Thus, it was the ethanol infusion/high level that treated the patient without causing any permanent organ damage, since the acute coma-related toxicities were mitigated in ICU. In this invention, the toxicity mitigation is planned ahead of time and monitored much better, so the safety is even higher.

In a modification of the above treatment model, isopropanol (or isopropyl alcohol) is infused in addition to ethanol. Isopropanol is also a commonly used alcohol for disinfection and deactivates many viruses, including the HIV virus (6, 7, 10). Since the above model aims to achieve an ethanol serum level of 400 mg/dl (the human comatose level), even though higher levels under anesthesia are achievable, the addition of a second alcohol increases the combined concentration of all alcohols taken together and their antiviral effect, but it does not add much to toxicity. Isopropanol is converted to acetone by alcohol dehydrogenase, an enzyme produced in the body. Acetone generates only small amounts of ketones and acidosis and it is less toxic than isopropanol itself, but it adds acidosis to toxicity; thus, this model needs monitoring of the blood pH and uses sodium bicarbonate infusions if acidosis becomes a problem, in clinically known rates of infusions as done in common clinical practice. The serum elimination half-life of isopropanol is usually 2.5 to 8 hours; this is known by toxicology data; but in the presence of ethanol which inhibits its metabolism, this half-life can increase up to 24-28 hours (7, 26, 27); so, in the presence of ethanol infusion, only one IV bolus of isopropanol is needed and the effects last during the entire treatment time of 24 hours; also, by blocking alcohol dehydrogenase (7), ethanol decreases the production of acetone and acidosis coming from isopropanol metabolism, making the acidosis even less important as a side effect of isopropanol. The dose of isopropanol used is 4 g/kg slow IV injection over 60 minutes, which is the median lethal dose in most animal species (7), but not lethal in the controlled setting of this model. The toxic effects of isopropanol are similar to the ones of ethanol: sedation, coma, hypoglycemia, hypotension, hypovolemia (6, 7, 24), and these are mitigated in the same way as those of ethanol (see above), but the antiviral effects are at least additive with ethanol when administered together, because the total blood concentration of alcohols is higher than in the first model above. The same monitoring of viral load is done here as above over 6 months, to confirm the full eradication of HIV virus (or other viruses in similar cases). The use of isopropanol in this model is a new drug indication for this substance, never used in the past.

Another modification of the isopropanol model can be the use of only small amount of ethanol, just to block the metabolism of isopropanol, and then use isopropanol single injection as the main treatment alcohol for HIV or other viruses. Isopropanol can achieve easier than ethanol concentrations in the blood below toxicity levels that are closer to in vitro disinfection levels, due to differences in molecular weight vs ethanol. This can make isopropanol more promising in the future. The principal toxicity of acute isopropanol poisoning is central nervous system depression (24). As with the ethanol-only model, this depression is unlikely to cause any harm in a patient who has already been anesthetized, airway protected, pre-oxygenated, and fluids administered before the infusion of isopropanol (see above).

A 3rd modality of the treatment model of this invention includes the infusion of ethanol as above plus another alcohol: methanol; the anesthetic model is similar to above; a 4th modality includes the infusion of ethanol with ethylene glycol; these alcohols have industrial applications and methanol is used at times in localized injections to kill tumors and parasites, but not in systemic injections (5, 6, 7, 8, 24, 27).

Methanol and ethylene glycol themselves have similar effects like ethanol above and are mitigated in the same way, but their metabolites cause severe acidosis and damage in multiple organs (8). That is why they are not used in general infusions in humans or animals for any treatment. In model 3 and 4, methanol and ethylene glycol, respectively, are each infused 3 hours after the ethanol infusion has started. This gives time to ethanol to block the alcohol dehydrogenase, the body enzyme that metabolizes these alcohols initially and is part of the pathway to generate toxic metabolites.

Without the activity of this enzyme, these toxic alcohols are not really toxic, because their toxicity comes mainly from their metabolites produced by the enzyme activity (6, 8, 24, 27). Ethanol itself is used as an antidote during methanol or ethylene glycol poisoning, exactly for its ability to block this enzyme and prevent the creation of toxic metabolites of methanol or ethylene glycol (5). The infusion of methanol or ethylene glycol in each model is at a total dose of 200 mg/kg bolus over 1 hour and is adjusted later to achieve serum target concentrations of 20 mg/dL, according to toxicology limit data (8).

These levels are measured with gas chromatography during the treatment period or with end-expiratory mass spectrometry. Another inhibitor of the enzyme, fomepizole, can be administered in addition to ethanol, to block further the dehydrogenase; this is a known inhibitor of the enzyme also used in cases of poisoning (6, 8, 24, 27, 28), but here this is a new indication that increases the safety of models 3 and 4; it is given with a bolus dose of 15 mg/kg IV over 30 minutes, followed by a dose of 10 mg/kg 12 hours later (8). This provides additional protection to ethanol against the formation of toxic metabolites. All these measures in the current invention to prevent toxicities are necessary, because the patient entering the treatment is expected to recover uneventfully and is not subject to an acute incidental intoxication. Many of these measures are used commonly as treatments after the problems appear. The innovation in this invention, among other things, is that these measures are started before toxicity occurs, to prevent side effects of alcohols.

Other modifications of the above models can be ethanol plus methanol plus isopropanol plus ethylene glycol, plus or minus fomepizole; these can be in different combinations and amounts, and need to be tested for safety and results; always, whenever there is methanol or ethylene glycol used, an inhibitor of the dehydrogenase is needed, either ethanol or fomepizole, to prevent their toxicity; the combinations of different alcohols increase the total alcohol concentration and virucidal effects by decreasing in the same time the specific organ toxicity of each one (since less of each can be used), and may make possible to achieve much higher alcohol levels than when used alone. This is named model treatment 5.

Another model for the treatment of HIV or other viruses is model 6. This model uses the same general anesthetic model as tested in the previous models. But the virus inactivation is done by a gas, not an alcoholic substance: ethylene oxide gas. This is a sterilant used for sterilization of instruments; it destroys completely all live organisms including viruses (see also below) (6); the model includes the modification of the usual anesthesia machine with the addition of a dial and delivery system for ethylene oxide in desired concentrations together with the usual other anesthetic gases like nitrous oxide, oxygen and sevoflurane. This type of anesthesia machine has never been used in the past, because it contains an additional gas section for ethylene oxide. Ethylene oxide is toxic but small temporary concentrations can be found below the toxic animal or human levels. Ethylene oxide is a small molecule and has good diffusing capacity which is important to deactivate the HIV and other viruses. It alkylates DNA and RNA (6), which is important for antiviral activity. Ethylene oxide acute toxicity (which is the only important issue in this invention), is mainly central nervous system depression and treatment is supportive (6), which can be mitigated in the same ways as with alcohols. Irritant effects on mucosas (6) can be managed like with irritant effects seen in most inhalation anesthetic gases (4). This is a new medical use for ethylene oxide. This is model 6. A modification of this model is to use the treatment with one of the above alcohol models plus the ethylene oxide in order to kill the virus(es) with two different mechanisms from two different sources; this makes the treatment more effective and helps to decrease the need for high toxic levels of each substance, thus decreasing the side effects of each.

In model 7, hexavalent and trivalent chromium are used, which are toxic to viruses and bacteria and have the tendency to accumulate in lymphoid tissue (6) where viral reservoirs are present (3, 17, 18, 19, 21, 22). The doses and solutions of these compounds are not known and need to be determined by more data. Modifications to this model would be combined uses of chromium compounds with the above alcohols or ethylene oxide in a variety of combinations.

Model 8.

Patients who failed the above treatment models and the viral load has been detected 6 months or longer after treatment, can be treated with model 8. In this model, arsenic trioxide is added to one of the above single- alcohol or alcohol-combination models. One of the causes of these failures may be the inability of the alcohols or other HIV treatment methods to destroy the part of the virus incorporated into the cell genome, which is one of the common causes of failure of the usual antiretroviral therapy to cure HIV (3, 17, 18, 21, 22). Arsenic trioxide is a small molecule used successfully for treating acute promyelocytic leukemia by inducing apoptosis (6). During apoptosis, the viral particles are exposed to the extracellular medium and are susceptible to damage by high tissue alcohol concentrations that are induced during the model.

General anesthesia is induced in the usual way, then arsenic trioxide is infused in a total dose of 0.15 to mg/kg over 4 hours, as it is done for leukemia (6). The arsenic blood level is monitored with a target range of 0.5 to 2 μmol/L, which are known to be the most effective apoptotic levels (6). These levels are reached by adjusting the infusion rate. After the infusion is complete (at 4 hours), the alcohol infusions are used as in the previous models.

Why Ethanol is Important in the Above Models?

Data show that the reasons why the HIV treatments fail to provide a permanent cure is that: There is a small degree of viral replication that continues despite the usual antiviral therapy; when antiretroviral drugs are stopped, this replication increases and goes back to high levels very shortly thereafter. 2. There are immune memory cells with long lifespan that carry the virus and it is difficult to differentiate them from non-infected cells, in order to kill them. 3. Some virus stays dormant in cell genome and in other cells like nervous cells etc where antiviral therapy does not penetrate (3, 17, 18, 21, 22).

Ethanol has these advantages as infusion, and some of these belong to the other small molecules, too: Ethanol can penetrate cell lipid membranes, and diffuses well in all body fluids, so can reach the target in all cells and membranes, systems, and nuclei (5).

Alcohol was shown to increase the HIV-1 replication in human peripheral blood mononuclear cells (22); monocytes are important in HIV infection as reservoir. The replication induction during treatment with ethanol and other alcohols helps to make the HIV virus particles in reservoirs to be visible in body fluids where ethanol and other alcohols are exerting their virucidal effects with similar levels as the blood. The levels needed for this effect were shown to be similar to levels obtained by chronic low, average and high users, which are at the range of 400 mg/dl or less as used in this invention; so, the levels of the invention are sufficient to achieve this proliferative effect, plus the long timing of the treatment of 24 hours can help in assuring that there is some time for replication. Another study achieved the same results with concentrations of ethanol tested at 115, 345 and 690 mg/dl (22).

Alcohol was shown to increase the HIV DNA in peripheral blood lymphocytes and their proliferation in concentrations 300-790 mg/dl (14).

Ethanol was shown to strongly potentiate the apoptosis induced by HIV-1 proteins in primary human brain microvascular endothelial cells in concentrations equivalent to 79 mg/dl and 237 mg/dl, when tested (20); these concentrations are clinically not deadly but are also below the level used in this invention and indicate that the apoptotic effect of ethanol is another advantage for its use for HIV eradication in all human cells, especially in reservoirs, including the central nervous system.

Even though not used in vivo with such high concentrations, ethanol has been used for in vitro inactivation of HIV virus in human albumin and plasma products at concentrations 18,000 mg/dl, and even higher, and then filtered before using the product in vivo (12); no damage to the product was caused, despite these very high levels; these levels are not used in vivo, but they show some safety with ethanol if the coma-induced side effects are mitigated as in the model;

In surfaces, with the use of 70% v/v ethanol, which is much higher than the concentration used clinically when converted to mg/dl, the time of contact needed for the inactivation of HIV virus is 2 minutes, and in some conditions, can go up to 4 minutes (29). The invention uses a 24-hour contact time to ensure that all viral particles are inactivated in the body, because such high concentrations of ethanol cannot be achieved in humans or animals without causing cardiovascular depression and death. So, the timing of exposure must be longer in vivo vs in vitro in order to compensate the differences in concentrations. With isopropanol, due to the difference in molecular weight vs ethanol, the clinical concentrations in this invention can be achieved to be closer to the disinfectant concentrations.

Concentration changes in ethanol blood levels after the levels are reached during treatment.

The target level of ethanol in blood in the above first model is 400 mg/dl (see above). Ethanol has a simple kinetics, and if infusion and boluses are stopped, this level decreases by 15-18 mg/dl per hour in a steady pace in a relatively predictable manner (1, 5, 6, 9, 23, 25). As such, the future levels and recovery are fully predictable for the patient. So, after the patient has had this level enough time to achieve virus inactivation, to reach a level below 100 mg/dl, which is the usual intoxicated level limit for most people and when the patient can wake up almost normally, it will take about 16-20 hours, provided that anesthetics are also discontinued. This is one of the reasons that the treatment takes at least 24 hours and it may take even longer depending on individual differences in alcohol metabolism and blood levels reached initially.

Other Viruses

The above invention with the different treatment modalities can be used to eradicate other viruses from the human and animal body. There are many other viruses that have the tendency to stay in the body for decades in latent state and cause disease when activated or when they replicate (30). Examples are:

Ebstein-Barr virus that causes infective mononucleosis, can cause also lymphomas later in life and the eradication may be done with the same method as above.

Hepatitis c virus becomes chronic and needs special treatment to eradicate, may cause liver cirrhosis and liver cancer

Herpes zoster virus. This causes chicken pox in children and then stays dormant for decades and may cause shingles usually after the age of 60, which can have multiple exacerbations at times.

Other viruses than can be treated with this invention are viruses that have high mortality and cause acute death. One such example is Ebola virus. This virus has the tendency to spread in multiple body fluids and tissues and has high mortality (31). The small molecules used in this invention are appropriate because they also penetrate all body fluids. Treatments for this illness are very limited and this disease triggers epidemics. Infected patients who start having symptoms may be treated with the above method with a single treatment under anesthesia in order to destroy/eradicate or at least decrease the titers of the virus for the first weeks until the body produces its own antibodies. This can save many lives and it is cost-effective.

West Nile Virus encephalitis or encephalitis caused by other viruses can also be treated with the above methods in the acute stage when the symptoms are severe. When viral encephalitis is severe, the mortality rate is very high and few medications are available. The alcohols penetrate well into the central nervous system and are able to destroy these viruses there.

Other Uses

Ethanol and sometimes methanol are used for destroying the hepatocellular carcinoma lesions in the liver by injecting them in high concentrations (usually absolute alcohol) locally through local blood vessels. They are also used when there is single metastasis of liver cancer in adrenal glands or elsewhere with the same method. These are only local or regional infusions.

They sometimes have general side effects or pain and patients are given analgesics or even general anesthesia in order to decrease the pain and discomfort (5). The current invention can be used to treat the hepatocellular carcinoma because the above models can deliver high systemic concentrations of alcohols with mitigation of toxicity. This may be able to decrease the chances of other metastases of liver cancer; several treatments may be needed or to be used intermittently. As such, this invention can also be extended to treat metastatic cancers of different types.

Another use of the above invention can be blood disorders like leukemias; leukemias and lymphomas have high division rates (32). Alcohols have been shown to penetrate in higher concentrations in actively dividing cells (14), so they can cause more damage to tumor/cancer cells than to normal cells and, as such, can be used to differentially destroy these cells.

Another use of the invention can be parasites that have already settled in body tissues, like schistosomiasis, malaria, trichinellosis, cysticercosis, etc. These parasites are killed by alcohols and they have many locations in organs and muscles, so the use of the above models makes it possible to reach them wherever they are in the body.

Alcohols and ethylene oxide have also antibacterial effects (6). As such, the above models can be used in cases of septicemia and septic shock, especially when the patient is already in ICU and under general anesthesia. By using the infusions and mitigating toxicities, they can reach the bacteria in the blood stream and in other body fluid and organs. A great advantage of these agents vs common antibiotics is that there is no known resistance of bacteria to their effects like it occurs with antibiotics. Ethanol was shown to decrease the risk of bloodstream catheter- related infections when injected preventively in central venous catheters (5).

Pediatric Applications

The above model can be used in pediatric cases, as long as the infusions doses are adjusted to weight of the child. This is important treatment for pediatric AIDS/HIV and for other viruses and blood disorders of childhood. Ethanol intoxication in children is treated similarly like in adults (9). So, at least the ethanol model can be applied to children, after mitigating toxicity with treatment of respiratory depression, hypoglycemia, hypovolemia, hypothermia (9), in a preventive manner under general anesthesia.

Anesthesia Machine Modifications

In the above models, the invention describes one modification of the common anesthesia machines when ethylene oxide gas is used. This adds a gas dial, connections, cylinders for ethylene oxide, similar to other gases used and as an addition to oxygen, sevoflurane and nitrous oxide gas connections.

Another modification of anesthesia machine can be used with alcohol models. A device similar to the breath test analyzer can be added to the anesthesia monitors.

Usually the end-expiratory concentrations of anesthetic gases and CO2 are monitored continuously in current anesthesia machines. This invention adds another monitor for the end-expiratory concentration of ethanol, isopropanol, ethylene glycol, methanol, and ethylene oxide, to be used and added alone or in combination, depending on the model of treatment needed to be used. These devices can work with mass spectrometry like most other anesthetic determinations or other methods of determination. For ethanol the breath concentrations correlate well with blood concentrations of ethanol with a factor of around 2300 (23). These monitors will make the use of blood samples for levels of these agents unnecessary and make their numbers easy to monitor and continuously.

Pregnancy Applications

Ethanol is known to be used as a tocolytic agent, even though there are better agents for this, but this is a known human application and the safety has been demonstrated (5). As such, based on this invention, ethanol can also be used near the end of pregnancy if the mother is HIV positive or has any other deadly virus or organism that can infect the fetus, like Ebola, etc. By using one treatment with the above models, the fetus can be prevented from being born with HIV positivity which will be a lifelong disease. Also, the pregnancy is not put at risk because ethanol has already been tested and it prevents premature labor.

Ethanol penetrates well the placenta and can also reach good fetal concentrations (5) for reaching infectious agents that are already in the fetus.

Equipment Modification Due to this Invention

Anesthesia machine: the standard machine in this invention is modified by adding ethylene oxide administration delivery device with concentration measured in ppm and delivered to inspiratory air flow; also, a check for safety is added before entering patient's airway.

Mass spectrometer in this invention is modified to include measurements of carbon dioxide, nitrous oxide, sevoflurane, ethanol, methanol, isopropanol, ethylene glycol, ethylene oxide.

New Drugs for Medical Use in this Invention

Ethanol has new indications as a drug IV infusion and boluses, for management of HIV under general anesthesia and for treatment of bacterial infections, viruses, parasites, Ebola, metastatic cancers, through general infusion with toxicity mitigation strategies

Isopropanol, new drug, IV infusion drug, for the same as above

Ethylene glycol, new drug, IV infusion, as above indicated

Methanol, new drug, IV infusion, as above indicated

Ethylene oxide; gas; new drug; can be used as antiviral by inhalation or even IV infusion; the inhalation way is for killing HIV and viruses; can be used also as a general anesthetic

Fomepizole, can be used as antidote for alcohols during induced coma for HIV treatment

Arsenic trioxide can be used as apoptosis inducer during HIV treatment, to expose the virus to the antiviral effects of other drugs

Chromium compounds can penetrate lymphoid tissue and kill HIV

New drug: ethanol in combination with methanol as IV infusion bag, to be injected together, pre-mixed

New drug: ethylene glycol and ethanol IV infusion pre-mixed bag

New drug: ethanol, methanol, ethylene glycol, pre-mixed IV infusion

New drug combinations pre-mixed: fomepizole with alcohols or arsenic trioxide with the other IV drugs

Addition about ethylene oxide

Ethylene oxide is a flammable gas (33), similar to other gases that are used as anesthetics (4). In this invention, ethylene oxide is used for the treatment if HIV and other viruses due to its effects as a sterilant. On the other hand, its properties make it possible to be used as a general anesthetic or as an adjunct general anesthetic. And this is also a new indication for this gas, which is currently used in medicine only as sterilant and not as a drug.

Ethylene oxide dissolves easily in water, alcohol, and organic solvents (33). This is another characteristic that helps the diffusion in all body tissues and important to reach the viral location areas. Data from exposure of humans to ethylene oxide in workplace show no increase in mortality (33). In mice, exposure to 400 ppm for 14 days resulted in no death (33), while higher levels like 800 ppm were deadly in 4 hours. Also, when exposed to 100 ppm for 2 years, monkeys and mice did not have increased mortality (33). Thus, for the purpose of this invention, the use of 400 ppm inhaled ethylene oxide as a maximum concentration for up to 24 hours is unlikely to cause any problems as long as the patient is monitored.

The major metabolite of ethylene oxide is ethylene glycol (33), one of the alcohols used in this study. As such, the use of ethylene oxide as an anesthetic and virucidal can also produce another virucidal substance, the ethylene glycol. This alcohol, as seen above, is toxic only through production of metabolites, which can be blocked by ethanol infusion. So, a slow ethanol infusion can be used before the inhalation of ethylene oxide to increase the viral activity and safety of the model. Ethylene oxide can be measured through gas chromatography (34), including the blood levels, but in this invention it is preferred to have it continuously monitored by mass spectrometry in the end-expiratory air.

Other Uses of the Invention

The same model of treatment can be used for viruses that are problematic in animals or similar to humans and can be used in veterinary medicine. These include: Feline immunodeficiency virus (FIV), a virus that affects cats with permanent infection of the cat that leads to death overtime like human HIV

Simian Immunodeficiency Virus (SIMV) for Monkeys

Other animal viruses that either harm animals after infecting the animal permanently without clearance or they have high acute phase mortality that makes the use of the invention a reasonable choice.

References

The American Psychiatric Publishing Textbook of Substance Abuse Treatment. Galanter M., Herbert K. D., Editors, 4th Edition, Washington, D.C., American Psychiatric Publishing, Inc. 2008.

HIV/AIDS Primary Care Guide. Steinhart C. et al. Editors, Gainesville, Fla., University of Florida, 2002.

Targeting Persistent HIV Reservoirs, PAR-15-041, Grant Program Announcement, National Institutes of Health at NIH.GOV, accessed April/2015.

Anesthesia. Miller, R D et al., Editors, 5th Edition, New York, N.Y., Churchill Livingstone, 2000.

Ethanol by Micromedex at http://www.micromedex.solutions.com, accessed April/2015

Goldfrank's Toxicologic Emergencies. Nelson L. S. et al. Editors, 9th Edition, New York, N.Y., McGraw Hill Medical, 2011.

Isopropyl Alcohol Poisoning at www.uptodate.com, accessed March/2015.

Methanol and Ethylene Glycol Poisoning at www.uptodate.com, accessed March/2015.

Ethanol Intoxication in Children, Clinical Features, Evaluation, and Management at www.uptodate.com, accessed March/2015.

Hanson P J, Gor D, Jeffries D J, Collins J V. Chemical inactivation of HIV on surfaces. BMJ. 1989 Apr. 1; 298(6677): 862-4.

de Jong J C, van Klingeren B. Chemical inactivation of HIV on surfaces. BMJ. 1989 Jun. 17; 298(6688): 1646-7.

Kim S., Eo H. G., Park C. W., et al. Removal and Inactivation of Human Immunodeficiency Virus (HIV-by Cold Ethanol Fractionation and Pasteurization during the Manufacturing of Albumin and Immunoglobulins from Human Plasma. Biotechnol. Bioprocess. Eng. 2001, 6: 25-30

Bienzle D. FIV in cats-a useful model of HIV in people? Vet Immunol Immunopathol. 2014 Jun. 15; 159(3-4): 171-9.

Liu X, Zha J, Nishitani J, Chen H, Zack J A. HIV-1 infection in peripheral blood lymphocytes (PBLs) exposed to alcohol. Virology. 2003 Mar. 1; 307(1): 37-44.

Silverstein P S, Kumar A. HIV-1 and alcohol: interactions in the central nervous system. Alcohol Clin Exp Res. 2014 March; 38(3): 604-10.

Wu E S, Metzger D S, Lynch K G, Douglas S D. Association between alcohol use and HIV viral load. J Acquir Immune Defic Syndr. 2011 Apr. 15; 56(5):e 129-30.

Fois A F, Brew B J. The Potential of the CNS as a Reservoir for HIV-1 Infection: Implications for HIV Eradication. Curr HIV/AIDS Rep. 2015 Jun.; 12(2): 299-303.

De Crignis E, Mahmoudi T. HIV eradication: combinatorial approaches to activate latent viruses. Viruses. 2014 Nov. 21; 6(11): 4581-608.

Bagasra O, Kajdacsy-Balla A, Lischner H W, Pomerantz R J. Alcohol intake increases human immunodeficiency virus type 1 replication in human peripheral blood mononuclear cells. J Infect Dis. 1993 April; 167 (4): 789-97

Acheampong E, Mukhtar M, Parveen Z, Ngoubilly N, Ahmad N, Patel C, Pomerantz R J. Ethanol strongly potentiates apoptosis induced by HIV-1 proteins in primary human brain microvascular endothelial cells. Virology. 2002 Dec. 20; 304 (2): 222-34.

Abbas W, Tariq M, Iqbal M, Kumar A, Herbein G. Eradication of HIV-1 from the macrophage reservoir: an uncertain goal? Viruses. 2015 Mar. 31; 7 (4): 1578-98.

Chen H, George I, Sperber K. Effect of ethanol on monocytic function in human immunodeficiency virus type 1 infection. Clin Diagn Lab Immunol. 1998 Nov.; 5 (6): 790-8.

Alcohol Toxicology for Prosecutors. American Prosecutors Research Institute at http://www.ndaa.org/pdf/toxicology_final.pdf accessed June/2015.

Dreisbach's Handbook of Poisoning. True B., Dreisbach R. H., Editors, 13th Edition, Boca Raton, FL, The Parthena Publishing Book, 2002.

Principles of Addiction Medicine. Ries R. K., Miller S. C., et al., Editors, 4th Edition, Philadelphia, Pa., Lippincott Williams & Wilkins, 2009.

Gaudet M P, Fraser G L. Isopropanol ingestion: case report with pharmacokinetic analysis. Am J Emerg Med. 1989 May; 7 (3): 297-9.

Bekka R, Borron S W, Astier A, Sandouk P, Bismuth C, Baud F J. Treatment of methanol and isopropanol poisoning with intravenous fomepizole. J Toxicol ClinToxicol. 2001; 39(1): 59-67.

Critical Care Medicine. Parrillo J. E., Dellinger R. P., Editors, 2nd Edition, St Louis, Mich., Mosby, 2001.

Van Bueren J, Cooke E M, Mortimer P P, Simpson R A. Inactivation of HIV on surfaces by alcohol. BMJ. 1989 Aug. 12; 299 (6696): 459.

Viral Pathogenesis. Nathanson N., Ahmed R., et al., Editors, Philadelphia, Pa., Lippincott-Raven, 1997.

Ebola, Centers for Disease Control and Prevention. At cdc.gov, accessed May/2015.

Manual of Clinical Oncology. Casciato D., A., Lowitz B. B., Editors, 4th Edition, New York, N.Y., Lippincott Williams & Wilkins, 2000.

Toxicological Profile for Ethylene Oxide. Agency for Toxic Substances and Disease Registry, US Public Health Service. Published December/1990.

NIOSH Manual of Analytical Methods (NMAM), 4^{th} Edition, August/1994.

What is claimed is:

1. A method of treating systemic infectious and neoplastic diseases in humans or animals, comprising the steps:
a. as a first step, placing the human or animal subject under standard general anesthesia, with inhalation and intravenous anesthetics and endotracheal intubation;
b. monitoring continuously life functions in standard general anesthesia practice;
c. injecting or giving by inhalation, using measured rates and concentrations, one or more of chemicals with small-sized molecules from the list of ethanol, isopropyl alcohol (isopropanol), methanol, and ethylene glycol, or that destroy infectious agents or neoplastic cells in vitro, and not to be classical antibiotics, and able to easily penetrate all body fluids and cells, in order to reach and destroy these agents wherever they are, without the need for using complex methods of separating infected (or abnormal) cells with non-infected (or healthy) cells.
d. monitoring the toxicity of these chemicals to the treated subject and monitoring the disease-killing ability, by monitoring directly or indirectly the blood levels of these chemicals and the vital functions;
e. controlling the toxicity of these chemicals to the treated subject by maintaining endotracheal intubation to prevent aspiration and provide ventilation, combining these chemicals in such a way as to maximize the disease-killing effect but minimize the toxicity to the subject, or in such a way as one chemical blocks the toxicity of the other(s);
f. continuing the infusion in sufficient time to destroy the disease agent(s) wherever they are found in the body; and
g. stopping the infusions and help the subject recover from general anesthesia as a last step.

2. The method in claim 1 wherein small virus-killing diffusible molecules can be used for permanently eradicating from the body the viruses human immunodeficiency virus, feline immunodeficiency virus, herpes viruses, ebola virus, and hepatitis c virus and other viruses.

3. The method in claim 1 wherein small virus-killing diffusible molecules can be used to suppress the replication of the virus human immunodeficiency virus, feline immunodeficiency virus, herpes viruses, ebola virus, hepatitis c virus and other viruses, even without permanent eradication.

4. The method in claim 1 wherein the above chemicals can be used to kill all kinds of bacteria in the body during systemic infections septicemia, sepsis, septic shock, bacterial endocarditis.

5. The method in claim 1 wherein the above chemicals can be used to kill parasites in the body including schistosomiasis, malaria, cysticercosis, and trichinellosis.

6. The method in claim 1 wherein the above chemicals can be used to kill cancer cells, lymphoma cells, tumors, and metastatic cells wherever they are in the body.

7. The method in claim 1 wherein the above treatment is used as a preventive single- or multiple-session procedure to prevent metastatic spread after the original tumor or cancer has been removed or treated.

8. The method in claim 1 wherein ethanol is the small disease-treating molecule used as an intravenous infusion.

9. The method in claim 8 wherein isopropyl alcohol (isopropanol) is added to the infusion of ethanol, thus achieving higher combined alcohol concentrations and better disease-treating activity, without significantly increasing toxicity, with ethanol blocking the toxicity of isopropanol.

10. The method in claim 8 wherein either methanol or ethylene glycol or a combination of those, are infused after the ethanol infusion is started, thus achieving higher combined alcohol concentrations and better disease-treating activity, without significantly increasing toxicity, with ethanol blocking the toxicity of methanol and ethylene glycol.

11. The method in claim 10 wherein fomepizole is added as an intravenous infusion in order to block the dehydrogenase enzyme activity in addition to ethanol and thus decreasing the toxicity of methanol and/or ethylene glycol.

12. The method in claim 1 wherein ethylene oxide gas is delivered by inhalation through a modified anesthesia machine, in order to kill viruses, bacteria and parasites.

13. The method in claim 1 wherein a variety of combinations of concentrations of ethanol, methanol, isopropanol, ethylene glycol, and ethylene oxide, are used in such way as to increase the combined activity against infectious or neoplastic diseases, but without increasing the toxicity to the subject.

14. The method in claim 1 wherein hexavalent or trivalent chromium is used with intravenous infusion in order to kill human immunodeficiency virus or other viruses.

15. The method in claim 2 wherein arsenic trioxide is injected as intravenous infusion before the other infusions, in order to induce apoptosis (cell death) and thus expose the intracellular virus or viruses to the killing effects of the chemicals with small-sized molecules.

16. The method in claim 3 wherein arsenic trioxide is injected as intravenous infusion before the other infusions, in order to induce apoptosis (cell death) and thus expose the intracellular virus or viruses to the killing effects of the chemicals with small-sized molecules.

17. The method in claim 8 wherein ethanol is used, by intravenous infusion, near the end of pregnancy, in human-imunodeficiency-virus-infected mother (human or animal), in order to eradicate the virus in mother and fetus and prevent the child from being born infected with the virus or getting infected during delivery.

18. The method in claim 1 wherein flumazenil is added as an intravenous infusion to reduce the depth of coma induced by alcohols and other small toxic molecules, and thus decrease the toxicity and make the subject's recovery from general anesthesia faster.

19. An anesthesia monitoring apparatus comprising
a. an expiratory air flow tube going from the patient to the anesthesia machine;
b. a thinner connection tube sampling the air of the said expiratory air flow tube;
c. a mass spectrometer device that measures continuously the end-tidal concentrations of ethanol, methanol, isopropanol, ethylene glycol, and ethylene oxide, in addition to the commonly monitored concentrations of carbon dioxide, nitrous oxide, and sevoflurane, and receives the air from the said connection tube; and
d. a monitoring screen where the concentrations of all of the said substances are displayed live over time.

20. An anesthesia air circuit device comprising:
a. a standard oxygen supply with meter and dial;
b. a standard nitrous oxide supply with meter and dial;
c. a common tubing that mixes the gases from the said meters and dials and sends the air mixture towards the patient;
d. a standard sevoflurane delivery device connected to the said tubing in the direction of the patient, and adding sevoflurane to the mixture;
e. an additional device connected to the said tubing in the direction of the patient, that measures and provides the desired concentrations of ethylene oxide gas to the air mixture, with the purpose of treating disease by killing infectious agents and for providing general anesthesia;
f. an ethylene oxide concentration final checking device with alarm system connected to the said tubing in the direction of the patient, with the purpose of safety; and
g. an oxygen concentration safety check device connected to the said tubing just before it connects to the endotracheal tube of the patient.
